(19) United States
(12) Patent Application Publication
(10) Pub. No.: US 2017/0143351 A1
(43) Pub. Date: May 25, 2017

(54) IMPLANT PLACEMENT TREPHINE, PREPACKAGED AND SIZED IMPLANT / TREPHINE KIT, AND METHODS OF USE

(71) Applicant: Nelorussi Corporation, Boston, MA (US)

(72) Inventors: Rustam K. DEVITRE, Boston, MA (US); Neal MACKENZIE, Pembroke, MA (US)

(21) Appl. No.: 15/357,081

(22) Filed: Nov. 21, 2016

Related U.S. Application Data
(60) Provisional application No. 62/258,951, filed on Nov. 23, 2015.

(51) Int. Cl. A61B 17/16; A61C 1/08; A61B 17/17; A61C 8/00

(52) U.S. Cl. CPC A61B 17/1637; A61C 8/0089; A61C 1/085; A61C 1/084; A61B 17/176

(57) ABSTRACT

An implant placement trephine may be used to drill into bone and also to bore a core of bone for purposes of forming an implant channel for implant placement. The trephine generally includes a trephine body with a cutting blade at one end and a spindle extending through the trephine body with a pilot drill at one end extending beyond the cutting blade. The spindle may be adjustably attached to the trephine body to allow the pilot drill to be adjusted relative to the trephine cutting blade. The trephine may be prepackaged with an implant, such as a dental implant, having a corresponding size. The implant placement trephine may be used for different osteotomy applications, for example, to form a channel for dental implants or orthopedic retaining stabilizers. The trephine may also facilitate harvesting bone from the core of bone removed for an autograft at the implant site.

### CROSS-REFERENCE TO RELATED APPLICATIONS

This application claims the benefit of co-pending U.S. Provisional Patent Application Ser. No. 62/258,951 filed on Nov. 23, 2015, which is fully incorporated herein by reference.

### TECHNICAL FIELD

The present disclosure relates to medical implant instruments and procedures and more particularly, to an implant placement trephine, a prepackaged and sized implant/trephine kit, and methods of use.

### BACKGROUND INFORMATION

Various medical procedures involve placement of implants into bone including, for example, dental implants and orthopedic retaining stabilizers (e.g., spinal and cranial stabilizers). Conventional placement of implants into bone of a patient typically involves the use of multiple drills to prepare a channel in the bone to secure an appropriate sized implant fixture. One or more pilot drills may be used, for example, before using a trephine to cut the bone. Surgical kits often include several sizes of implant drills to be used in series during an implant placement procedure. The selection of the drills during the procedure depends, at least in part, on the size of the implant being placed. As such, multiple drills increase the complexity and duration of the procedure and may cause confusion, particularly for novices. Furthermore, cleaning of the multiple drills between procedures can be time consuming and presents risk of cross contamination.

### SUMMARY

Consistent with an embodiment, an implant placement trephine includes a shaft configured to be engaged by a rotary driving tool, a trephine body coupled at one end to the shaft, and a spindle adjustably coupled at one end to the trephine body. The trephine body includes a cutting blade at an opposite end and defines a cylindrical region proximate the cutting blade. The spindle includes a pilot drill at an opposite end, which extends beyond the cutting blade on the trephine body. The spindle is also adjustable relative to the trephine body to adjust an extent to which the pilot drill extends beyond the cutting blade.

Consistent with another embodiment, a prepackaged sized dental implant kit includes a dental implant having a predefined implant size and a disposable implant replacement trephine having a predefined trephine size corresponding to the predefined implant size. The disposable implant replacement trephine includes a shaft configured to be engaged by a driving tool, a trephine body coupled at one end to the shaft, and a spindle coupled at one end to the trephine body. The trephine body includes a cutting blade at an opposite end and defines a cylindrical region proximate the cutting blade. The spindle includes a pilot drill at an opposite end, which extends beyond the cutting blade on the trephine body.

Consistent with a further embodiment, method includes: providing a dental implant and an implant placement trephine sized to correspond to the dental implant, the implant placement trephine including a trephine body with a cutting blade defining a cylindrical region and a spindle with a pilot drill extending beyond the cutting blade; aligning the pilot drill with a furcation region on an intraradicular bone in an empty tooth socket; rotating the pilot drill and trephine body together to drill into the intraradicular bone with the pilot drill; boring a core around the intraradicular bone with the cutting blade to form an implant channel; harvesting bone from the core; placing the dental implant into the implant channel; and grafting with the harvested bone within the empty tooth socket to form a grafted implant site.

### BRIEF DESCRIPTION OF THE DRAWINGS

These and other features and advantages will be better understood by reading the following detailed description, taken together with the drawings wherein:

FIG. 1 is an exploded perspective view of an implant placement trephine, consistent with an embodiment of the present disclosure.

FIG. 2 is a perspective view of the assembled implant placement trephine shown in FIG. 1.

FIG. 3 is a side view of a rotary driving tool with the implant placement trephine shown in FIG. 2.

FIG. 4 is a side view of the implant placement trephine shown in FIG. 2.

FIG. 5 is a cross-sectional view of the implant placement trephine taken along line 5-5 in FIG. 4.

FIG. 5A is an enlarged view of the cutting teeth shown in section A in FIG. 5.

FIG. 5B is an enlarged perspective view of the cutting teeth at the end of the trephine body shown in FIG. 1.

FIG. 5C is an enlarged end view of the cutting teeth at the end of the trephine body shown in FIG. 1.

FIG. 6 is a side view of a prepackaged sized dental implant kit including a placement implant trephine and a dental implant of a corresponding size, consistent with another embodiment of the present disclosure.

FIG. 7 is a side view of another embodiment of an implant placement trephine including markings for indicating depth of cut.

FIGS. 8A-8F illustrate a dental implant procedure using an implant placement trephine, consistent with an embodiment of the present disclosure.

### DETAILED DESCRIPTION

An implant placement trephine, consistent with embodiments described herein, may be used to drill into bone and also to bore a core of bone for purposes of forming an implant channel for implant placement. The implant placement trephine generally includes a trephine body with a cutting blade at one end and a spindle extending through the trephine body with a pilot drill at one end extending beyond the cutting blade. The spindle may be adjustably attached to the trephine body to allow the pilot drill to be adjusted relative to the trephine cutting blade. The implant placement trephine may be prepackaged with an implant, such as a dental implant, having a corresponding size. The implant placement trephine may be used for different osteotomy applications, for example, to form a channel for dental implants or orthopedic retaining stabilizers. The implant placement trephine may also facilitate harvesting bone from the core of bone removed for an autograft at the implant site.

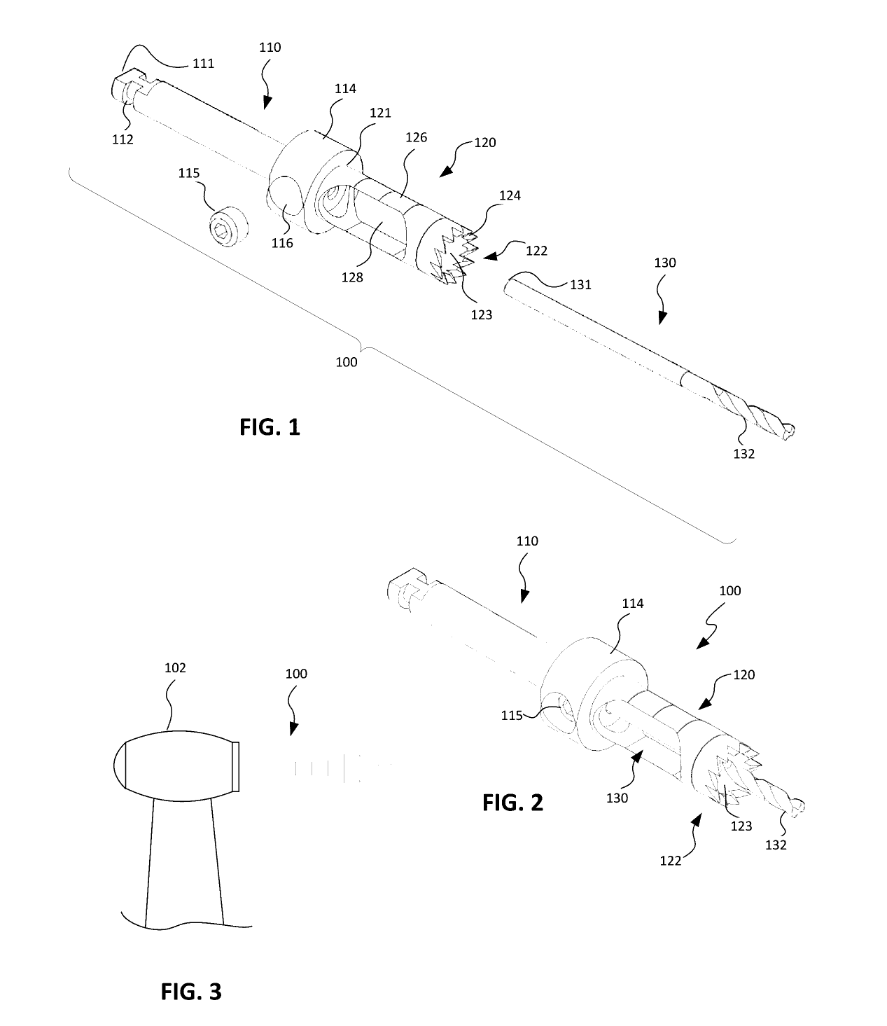

Referring to FIGS. 1-5, one embodiment of an implant placement trephine 100 is shown and described in detail. In general, the implant placement trephine 100 includes a shaft 110, a trephine body 120 coupled at one end 121 to the shaft 110, and a spindle 130 adjustably coupled at one end 131 to the trephine body 120. The illustrated embodiment of the implant placement trephine 100 may be used for drilling into bone and boring a core of bone for dental implants, as will be described in greater detail below. Other variations of an implant placement trephine consistent with the present disclosure and using the concepts described herein may be used in other osteotomy applications.

The shaft 110 may be configured to be engaged by a rotary driving tool 102 for rotating the trephine 100, for example, as shown in FIG. 3. The rotary driving tool 102 may include, for example, a surgical handpiece known for use in dentistry. In the illustrated embodiment, for example, the shaft 110 includes an engagement end 111 (e.g., including a contra-angle notch 112) that allows engagement with surgical handpieces.

The trephine body 120 may be formed as one piece with the shaft 110 or may be formed separately and attached to the shaft 110, for example, by welding or soldering. The trephine body 120 includes a cutting blade 122 at an end opposite the end coupled to the trephine shaft 110. The trephine body 120 defines a cylindrical region 123 proximate the cutting blade 122, which receives a core of bone as the cutting blade 122 cuts into the bone. The cutting blade 122 has longitudinally extending teeth 124 extending generally along a longitudinal axis of the trephine body 120 and positioned annularly around the cylindrical region 123, as will be described in greater detail below. In other embodiments, the cutting blade 122 may also include one or more teeth extending generally radially and/or at an angle relative to the longitudinal axis.

The illustrated embodiment of the trephine body 120 also includes sides 126 and a transverse open region 128 between the cutting blade 122 and the shaft 110. The sides 126 have a width sufficient to support the cutting blade 122 (e.g., at least about 1 mm wide). The trephine 100 may be made of a metal such as medical grade stainless steel (e.g., 316 or 17-4 stainless steel).

In an embodiment, the trephine body 120 includes a drill stop 114 proximate the end 121 coupled to the shaft 112. The drill stop 114 may be used to determine when to stop advancing the cutting blade 122 during drilling. In the illustrated embodiment, the drill stop 114 extends annularly around the trephine body 120 between the trephine body 120 and the shaft 110. The drill stop 114 may also include other structures having different shapes.

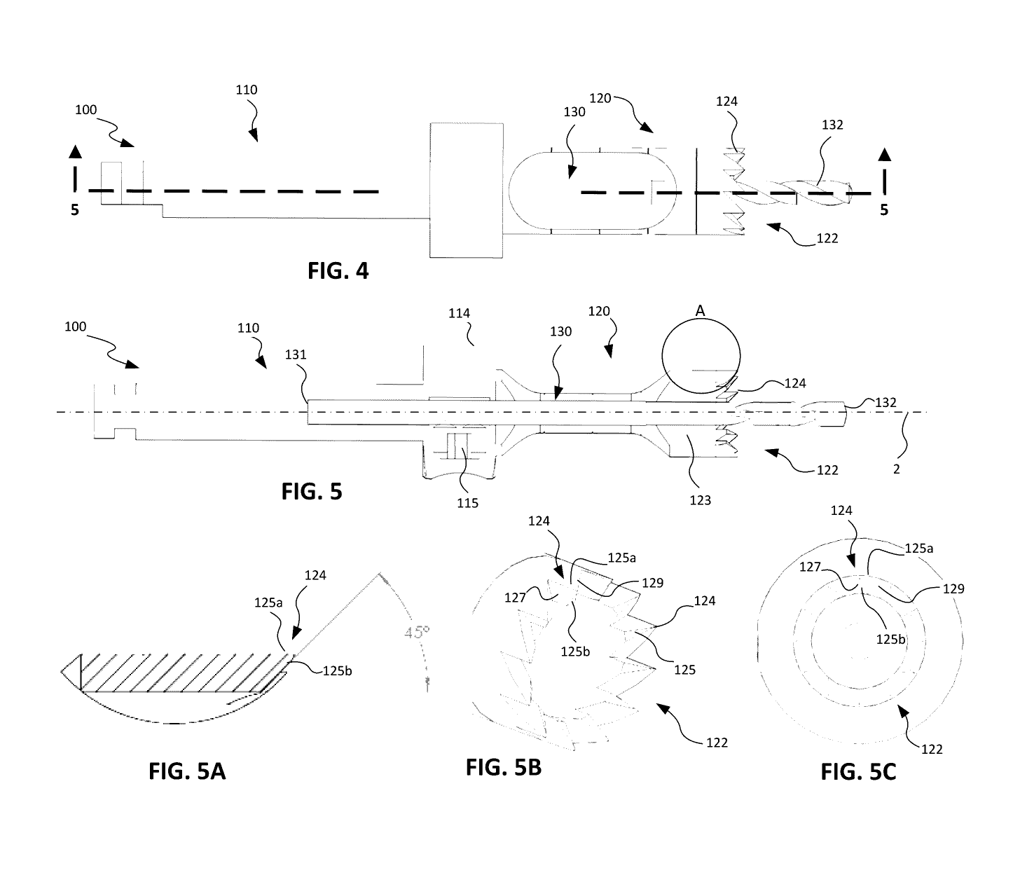

The spindle 130 includes a pilot drill 132 at an end opposite the end 131 coupled to the trephine body 120 and provides stability while drilling. As shown in FIGS. 2 and 4, the pilot drill 132 extends beyond the cutting blade 122. The other end 131 of the spindle 130 is inserted into a longitudinal bore in the trephine body 120 and may extend into the shaft 110, as shown in FIG. 5. The spindle 130 may be adjustably coupled, for example, using a set screw 115 threaded within a side bore 116 and tightened against the spindle 130. The extent to which the pilot drill 132 extends beyond the cutting blade 122 may thus be adjusted, for example, by loosening the set screw 115 and moving the spindle 130 relative to the trephine body 120. The spindle 130 may also be adjusted such that the pilot drill 132 is retracted inside the cutting blade 122 of the trephine body 120 or removed entirely. In one example, the spindle 130 may be adjustable to provide a range of extension to about 3 mm beyond the cutting blade 122 and retraction to about 2 mm inside the cutting blade 122. As used herein, “about” means the same or within acceptable tolerances. Other coupling mechanisms may also be used to couple the spindle 130 to the trephine body 120 such as other types of screws or fasteners.

Referring to FIGS. 5A-5C, the longitudinally extending cutting teeth 124 are described in greater detail. One or more of the teeth 124 includes an outside longitudinal face 125a extending substantially parallel to the longitudinal axis 2, radial faces 127, 129 extending radially relative to the longitudinal axis 2, and an inside angled face 125b extending at an angle relative to the longitudinal axis 2. In the illustrated embodiment, as shown in FIG. 5A, the inside angled face 125b is at an angle of 45°. The radial face 127 lies in a plane substantially parallel to the longitudinal axis 2 and the radial face 129 lies in a plane that is angled relative to the longitudinal axis 2. This design of the cutting teeth 124 facilitates cutting the bone and boring a core that may be harvesting for autografting.

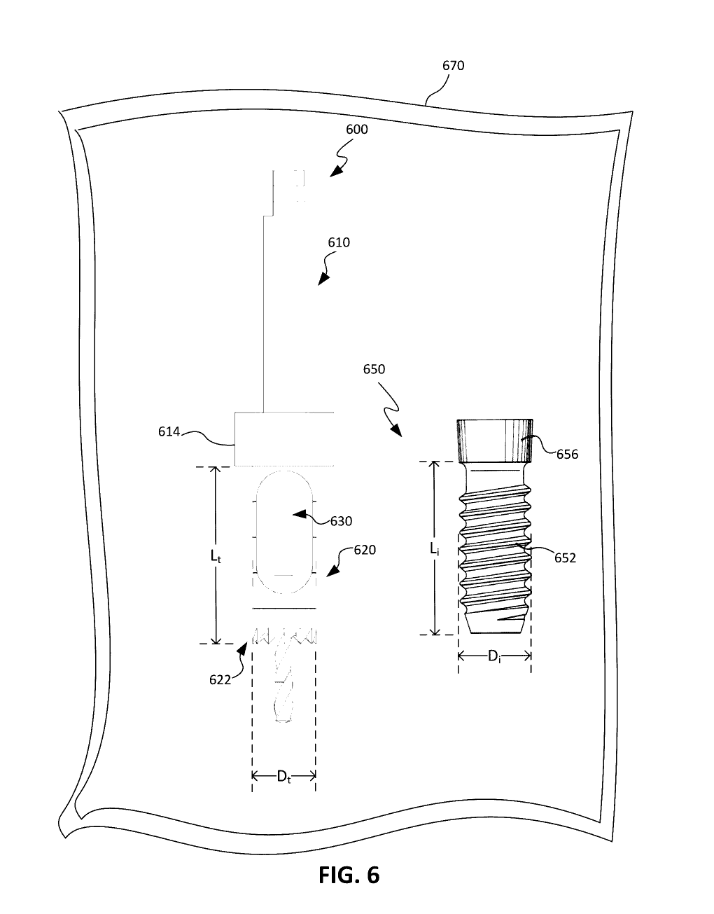

As shown in FIG. 6, one embodiment of an implant placement trephine 600 has a trephine body 620 sized to correspond to a dental implant 650. The dental implant 650 may be a root form implant, which replaces the root of a missing tooth. The dental implant 650 includes at least an implant fixture 652 configured to be threaded or otherwise implanted into a bore formed in the bone to replace the root of the tooth. In the illustrated embodiment, an abutment or post 656 extends from the implant fixture 652 and is configured to receive a crown (not shown) shaped like the missing tooth. The post 656 may be formed as one piece with the fixture 652 or may be a separate piece attached to the fixture 652 after the fixture 652 has been implanted. Although one example of a dental implant 650 is shown, other examples with different shapes and/or configurations are also within the scope of the present disclosure.

In general, the trephine body 620 is sized to correspond to the dental implant 650 in that the trephine body 620 is configured to form a bore of a size appropriate for receiving the fixture 652 of the implant 650. In one embodiment, an outer diameter Dₜ of a cutting blade 622 at the end of the trephine body 620 is smaller than an outer diameter Dᵢ of the implant fixture 652 such that the implant fixture 652 expands the bone and bites into the bone with a torque of at least about 45 N/cm. In particular, the outer diameter Dₜ of the cutting blade 622 may be about 0.4 to 0.5 mm smaller than the outer diameter Dᵢ of the implant fixture 652 and may range, for example, from 2.8 mm to 8.0 mm. Examples of dental implant sizes include diameters of 3.5 mm for front lower implants, 4.0 mm for incisors lateral, 4.5 mm for bicuspids, and 5.0, 5.5, and 6.0 mm for molars. Thus, the implant placement trephine 600 may create a hole of an ideal size for the corresponding dental implant 650 to provide the desired native bone stability for a successful implant.

The trephine body 620 may also have a length corresponding in size to the implant 650. The length Lₜ of the trephine body 620 from a stop 614 to the cutting blade 622 may be about the same as the length Lᵢ of the implant fixture 652 such that the stop 614 may be used to determine when the hole is drilled deep enough. Examples of dental implant sizes include lengths Lᵢ of about 8.5 mm, 10.0 mm, 11.5 mm, 13 mm and 16 mm. As such, trephines 600 of a corresponding size may have lengths Lₜ of about 8.5 mm, 10.0 mm, 11.5 mm, 13 mm and 16 mm.

The implant placement trephine 600 may be packaged together with the dental implant 650 of the corresponding size. Providing a single implant placement trephine 600 for use with a particular sized implant 650 avoids having to select from different sized drills and thus avoids confusion. The trephine 600 and dental implant 650 may be sterilized and packaged, for example, in sterile packaging 670 known for use with medical instruments. The implant placement trephine 600 may be packaged with a single dental implant (as shown) or multiple dental implants of the corresponding size. The prepackaged trephine 600 may be a single-use, disposable device, thereby saving time involved with sterilization and reducing the possibility of cross-contamination. Alternatively, the prepackaged trephine 600 may be re-sterilized and used multiple times with dental implants having a corresponding size.

Although the illustrated embodiment shows the trephine 600 and dental implant 650 in one package, the trephine 600 and the dental implant 650 may be provided in separate packages. The trephine may also be sold separately and the operator may select the appropriate sized trephine based on the selected implant design and size.

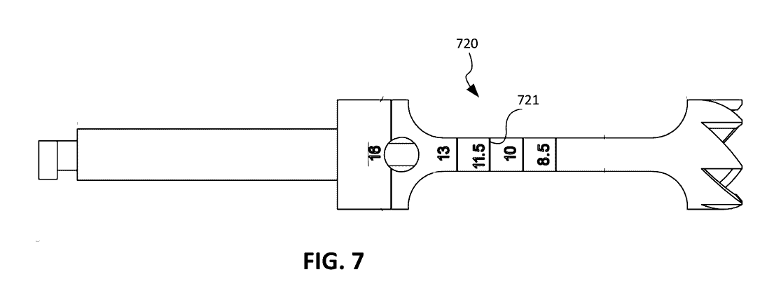

As shown in FIG. 7, another embodiment of a trephine 700 includes markings 721 on the trephine body 720 to determine a depth of the bore cut into the bone. In this embodiment, a trephine 700 having a trephine body with one length may be used with implants having different lengths. In this example, the markings 721 correspond to implant lengths of 8.5 mm, 10 mm, 11.5 mm, 13 mm, and 16 mm.

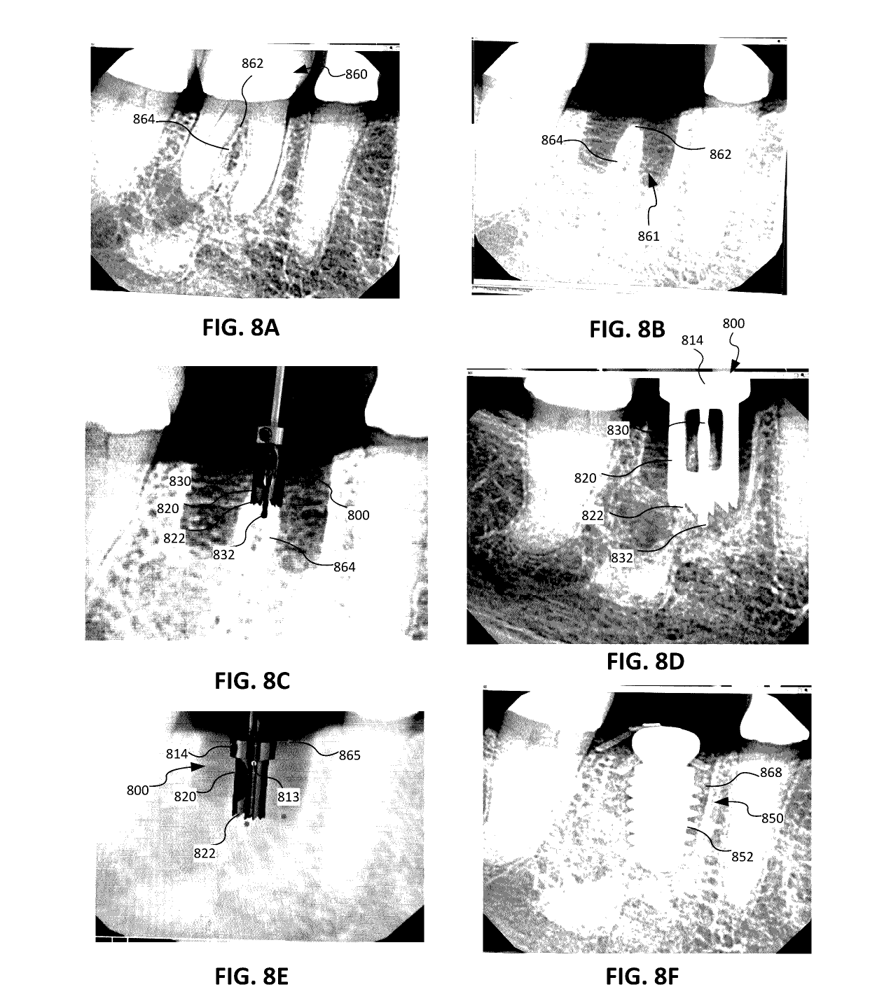

Referring to FIGS. 8A-8D, a method for of using the implant placement trephine, consistent with embodiments of the present disclosure, is shown and described in greater detail. The illustrated embodiment shows the implant placement trephine being used to develop a dental implant channel for placement of a dental implant in a lower molar region. The implant placement trephine may also be used for an upper molar region, an upper or lower bicuspid region, and an upper or lower anterior region. In general, the implant placement trephine bores a core of bone in the interdental/furcation region of the empty molar socket and creates a cylinder of bone that may be harvested for use as an autograft in the socket once the implant is inserted in the channel formed by harvesting the bone.

In the illustrated example method, a molar 860 (FIG. 8A) is extracted to form an empty tooth socket 861 including an intraradicular bone 864 having a furcation region 862 (FIG. 8B). An implant replacement trephine 800 and dental implant 850 are selected based on the dimensions of the empty tooth socket 861 and the intraradicular bone 864. A radiograph or CT scan may be used to determine the size of the dental implant 850 and the trephine 800 to be selected. In the illustrated example, the trephine 800 has a length from the drill stop 814 to the cutting blade of about 10 mm and an outer diameter of about 5.5 mm and the dental implant 850 has a length of about 10 mm and an outer diameter of about 6 mm. The pilot drill 832 is initially set to extend beyond the cutting blade 822 to provide alignment and stability during the initial cutting (FIG. 8C). In one example, the pilot drill 832 is set to protrude beyond the cutting blade 822 by about 2 mm; however, the protrusion may vary, for example, depending on where the nerve is located.

The pilot drill 832 of the trephine 800 is aligned with the furcation region 862 of the intraradicular bone 864 in the empty tooth socket 861. This alignment should result in the trephine body 820 and cutting blade 822 being aligned and positioned over the intraradicular bone 864, for example, as shown in FIG. 8C illustrating the trephine 800 superimposed on a radiograph showing the intraradicular bone 864. The trephine body 820 may also be appropriately spaced from the buccal crest and the proximal crest (e.g., by about 2 mm).

The trephine 800 is rotated with the rotary driving tool (e.g., at 1200 RPM) and together with saline such that the pilot drill 832 drills into the furcation region 862 while the cutting blade 822 of the trephine body 820 cuts around the crest of the intraradicular bone 864 to provide an initial cut and penetration into the intraradicular bone septum (FIG. 8D). Thus, the spindle 830 with the pilot drill 832 facilitates both alignment and stability during initial drilling. This initial penetration into the intraradicular bone septum may be, for example, about 5 mm. After an initial cut is made, the spindle 830 may be retracted such that the pilot drill 832 is located inside of the cutting blade 822 and cutting may be resumed with the pilot drill 832 and spindle 830 continuing to provide stability. Alternatively, the spindle 830 may be removed after the initial cut.

The subsequent cutting may be performed to a desired final depth for implant placement. A drill stop 814 and/or one or more markings (not shown) on the trephine body 820 may be used to gauge the proper depth of the cut for implant placement. Advancement of the trephine 800 may be stopped, for example, when the lower edge 813 of the drill stop 814 lines up with the lower of the buccal and lingual crest of the empty tooth socket 861 (FIG. 8E). In the illustrated example, the lower edge 813 of the drill stop 814 lines up with the buccal crest, which is lower than the lingual crest 865. Other depths are possible based on operator preference and/or the expected success of the graft. A radiograph may be taken to confirm the depth of the cut and the final osteotomy relative to the surrounding structures such as the mandibular nerve or the floor of the sinus wall.

The core of bone may be removed for use as an autograft. The core of bone may be removed using known instruments such as, for example, a molt curette, an osteotome or a periotome instrument. After removing the core, the apical end of the osteotomy may be made smooth, for example, using the pilot drill 832 or another instrument such as a long shank surgical burr/drill to round off the roughness caused by removing the core.

The implant fixture 852 may then be lowered into the channel, for example, at a surgical rotary speed in a range of 50 to 125 rpm. The implant fixture 852 may be lowered such than an implant face is below (e.g., about 2 mm) the lowest crest of bone in the socket to assure that implant threads are well-immersed in the bone. The harvested bone may then be combined with a particulate bone graft in the socket area 868 around the implant fixture 852 (FIG. 8D). The particulate bone graft may include a freeze-dried bone allograft (FDBA), a cadaver bone allograft, a xenograft from horse, cow, or pig bone, or a synthetic graft.

Other variations of this method for using the implant placement trephine are also within the scope of the present disclosure. For example, the implant placement trephine may be used to bore the core without retracting or removing the pilot drill.

Accordingly, an implant placement trephine, consistent with embodiments described herein, simplifies the process of implant placement by providing a single device sized for a particular implant and by facilitating alignment and drill stability. Prepackaging the implant placement trephine with an implant of a corresponding size further facilitates use and avoids cross-contamination when used as a disposable, single-use device. Moreover, the implant placement trephine may be used to both bore a core in the bone and harvest the core for an autograft.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

What is claimed is:

1. An implant placement trephine comprising:
a shaft configured to be engaged by a rotary driving tool;
a trephine body coupled at one end to the shaft and including a cutting blade at an opposite end, the trephine body defining a cylindrical region proximate the cutting blade; and
a spindle adjustably coupled at one end to the trephine body and including a pilot drill at an opposite end, wherein the pilot drill extends beyond the cutting blade on the trephine body, and wherein the spindle is adjustable relative to the trephine body to adjust an extent to which the pilot drill extends beyond the cutting blade.

2. The implant placement trephine of claim 1 wherein the cutting blade is an annular cutting blade defining the cylindrical region.

3. The implant placement trephine of claim 1 wherein the cutting blade includes longitudinally extending teeth.

4. The implant placement trephine of claim 2 wherein at least one of the longitudinally extending teeth includes an inside angled face extending at an angle relative to a longitudinal axis.

5. The implant placement trephine of claim 1 further including a set screw engaged with the one end of the spindle.

6. The implant placement trephine of claim 1 further including markings on the trephine body and the spindle for indicating depth of cut of the cutting blade.

7. The implant placement trephine of claim 1 further including a drill stop extending radially from the trephine body proximate the end coupled to the shaft.

8. The implant placement trephine of claim 1 wherein the spindle extends through the trephine body and into the shaft.

9. The implant placement trephine of claim 1 wherein the trephine body includes a transverse open region between the cutting blade and the shaft.

10. A prepackaged sized dental implant kit comprising:
a dental implant having a predefined implant size; and
a disposable implant replacement trephine having a predefined trephine size corresponding to the predefined implant size, the disposable implant replacement trephine including:
a shaft configured to be engaged by a driving tool;
a trephine body coupled at one end to the shaft and including a cutting blade at an opposite end, the trephine body defining a cylindrical region proximate the cutting blade; and
a spindle coupled at one end to the trephine body and including a pilot drill at an opposite end, wherein the pilot drill extends beyond the cutting blade on the trephine body.

11. The prepackaged sized dental implant kit of claim 10 wherein the cutting blade has an outer diameter that is about 0.4 to 0.5 mm less than an outer diameter of a fixture of the implant.

12. The prepackaged sized dental implant kit of claim 10 further including a drill stop extending radially from the trephine body proximate the end coupled to the shaft, wherein a length of the trephine body from the cutting blade to the drill stop is about the same as a length of a fixture of the implant.

13. The prepackaged sized dental implant kit of claim of claim 10 wherein the spindle is adjustable relative to the trephine body to adjust an extent to which the pilot drill extends beyond the cutting blade

14. The prepackaged sized dental implant kit of claim 10 further including markings on the trephine body and the spindle for indicating depth of cut of the cutting blade.

15. A method comprising:
providing a dental implant and an implant placement trephine sized to correspond to the dental implant, the implant placement trephine including a trephine body with a cutting blade defining a cylindrical region and a spindle with a pilot drill extending beyond the cutting blade;
aligning the pilot drill with a furcation region on an intraradicular bone in an empty tooth socket;
rotating the pilot drill and trephine body together to drill into the intraradicular bone with the pilot drill;
boring a core around the intraradicular bone with the cutting blade to form an implant channel;
harvesting bone from the core;
placing the dental implant into the implant channel; and
grafting with the harvested bone within the empty tooth socket to form a grafted implant site.

16. The method of claim 15 wherein the pilot drill is retracted or removed before boring the core around the intraradicular bone with the cutting blade.

17. The method of claim 15 wherein the empty tooth socket is located in a molar region or a bicuspid region.

18. The method of claim 15 wherein providing the dental implant and the implant placement trephine includes selecting a dental implant of an appropriate size for the empty tooth socket and using the implant placement trephine prepackaged with the dental implant of the appropriate size.

19. The method of claim 15 further comprising adjusting a distance that the pilot drill extends beyond the cutting blade.

20. The method of claim 15 further comprising advancing the rotating drill until a drill stop or marking on the trephine body is aligned with a crest proximate the empty tooth socket.
